(19) United States
(12) Patent Application Publication
(10) Pub. No.: US 2008/0269564 A1
(43) Pub. Date: Oct. 30, 2008

(54) Surgical retractor system and method

(75) Inventor: Mark Gelnett, Houston, TX (US)

Correspondence Address: VINSON & ELKINS L.L.P., First City Tower, 1001 Fannin Street, Suite 2300, HOUSTON, TX, 77002-6760, US

(73) Assignee: CooperSurgical, Incorporated, Trumbull, CT (US)

(21) Appl. No.: 12/148,186

(22) Filed: Apr. 17, 2008

Related U.S. Application Data
(60) Provisional application No. 60/923,849, filed on Apr. 17, 2007.

(51) Int. Cl. A61B 1/32

(52) U.S. Cl. 600/201

(57) ABSTRACT

The present invention provides a system and method of retracting tissue during medical procedures. In one embodiment, the present invention provides a blade portion for retracting tissue and a shaft portion coupled thereto. In one embodiment, the shaft portion of the surgical retractor is adapted to engage at least one retaining member. The retaining member may have one or more channels through which the shaft portion may be slidably inserted. In one embodiment, the retaining member provides one or more securing members for receiving at least one elastic member.

This patent application claims priority upon a provisional patent application entitled “Surgical Retractor System and Method,” filed on Apr. 17, 2007, Ser. No. 60/923,849.

### FIELD OF THE INVENTION

The present invention relates generally to medical devices and, more particularly, to a system and method of retracting tissue during surgical procedures.

### BACKGROUND OF THE INVENTION

During the course of many surgical procedures, anatomical features or tissue surrounding an incision or wound is separated and retracted by means of retractors or stay sutures, which are generally held by trained assistants. Most retractors are one piece metallic implements that retract a wound in a non-yielding manner. Many retractors retract the wound in a manner such that manipulation and movement by the surgeon as well as movement caused by contracting muscles or tissues of the patient can result in bruising or tearing of the tissue.

There remains a need for a surgical system capable of efficiently retracting and holding tissue segments without causing undue injury to such tissue.

### SUMMARY OF THE INVENTION

Accordingly, the present invention provides a system and method of retracting tissue during medical procedures. In one embodiment, the present invention provides a blade portion for retracting tissue and a shaft portion coupled thereto. In one embodiment, the blade portion of the surgical retractor may have one or more curved portions for gently retracting any number of tissue segment types and/or organs.

In one embodiment, the shaft portion of the surgical retractor is adapted to releasably engage at least one retaining member. The retaining member may have one or more channels through which the shaft portion may be slidably inserted. The retaining member may be releasably attached to a surgical drape, retractor plate, or any other surface suitable as a platform for surgical procedures. In one embodiment, the retaining member provides one or more securing members for receiving at least one elastic member. Further, the retaining member may provide an interlocking member, a fin member, and one or more protrusions to facilitate the releasable attachment of the retaining member to a retractor plate.

In one embodiment, the present invention utilizes one or more elastic members to provide a tensile force capable of holding tissue in a retracted position. In this embodiment, the first end of the elastic member is releasably or permanently attached to the blade portion of the surgical retractor and the second end is releasably or permanently attached to a securing member provided by the retaining member. This unique feature of the present invention allows medical personnel to adjust the amount of tension applied to retracted tissue during surgical procedures.

In one embodiment, the surgical retractor of the present invention has at least one angular portion having an angle of between about 45 degrees and about 150 degrees. The angular portion may be altered depending on whether a retractor plate is utilized. For example, when a retractor plate having a 20 degree tilt is utilized for the secure positioning of the retaining member, the angular portion may utilize an angle of about 100 degrees whereas, when no retractor plate is utilized, the angular portion may utilize an angle of about 120 degrees.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings; it being understood that the drawings contained herein are not necessarily drawn to scale; wherein:

FIG. 1 is a perspective view of the surgical retractor of one embodiment of the present invention.

FIG. 2 is a perspective view of the surgical retractor of one embodiment of the present invention.

FIGS. 2A-2C are elevation views illustrating the cross sectional configurations of the shaft of the surgical retractor in some example embodiments of the present invention.

FIG. 3 is a perspective view of the surgical retractor of one embodiment of the present invention shown in conjunction with a retractor plate.

FIG. 3A is a perspective view of the surgical retractor of one embodiment of the present invention.

FIG. 3B is a perspective view of the elastic member of one embodiment of the present invention.

FIG. 4 is a perspective view of a retaining member of one embodiment of the present invention shown in conjunction with a portion of a retractor plate.

FIG. 5 is a bottom view of a retaining member of one embodiment of the present invention shown in conjunction with a portion of a retractor plate.

FIG. 6 is a bottom view of a retaining member of one embodiment of the present invention releasably attached to a portion of a retractor plate.

FIG. 7 is a perspective view of a retaining member of one embodiment of the present invention.

FIG. 8 is a side view of a retaining member of one embodiment of the present invention.

FIG. 9 is a front view of a retaining member of one embodiment of the present invention.

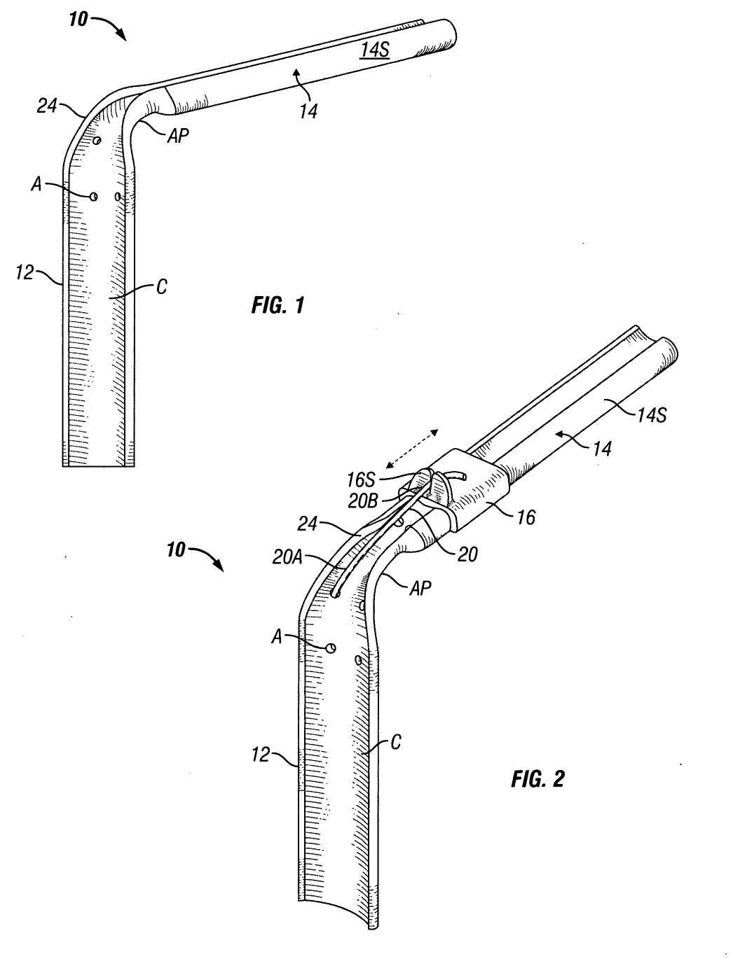

### DETAILED DESCRIPTION OF THE INVENTION

Referring to the Figures, the present invention is herein described as a surgical system and method. The present invention provides a surgical retractor (10) having a blade portion (12) for retracting tissue. In one embodiment, the blade portion of the surgical retractor is composed of a malleable metallic or plastic material capable of exerting sufficient tissue retraction force in preparation for and during surgical procedures. In one embodiment, the blade portion of the surgical retractor may have one or more curved portions (C) for gently retracting tissue and/or organs.

In one embodiment, the blade portion (12) of the surgical retractor may be coupled to a shaft portion (14). The shaft portion may be utilized, at least in part, to provide the surgeon or other medical personnel with a convenient grasping surface. Any suitable method of attaching the blade and shaft portions may be used, it being understood that such attachment may be permanent or temporary.

In one embodiment, the shaft portion of the surgical retractor is adapted to releasably engage at least one retaining member (16). In one embodiment, the retaining member may have one or more channels (16C) through which the shaft portion may be inserted. In one embodiment, at least one channel formed by an inner surface of the retaining member is adapted to slidably engage an outer surface (14S) of the shaft (14) of the surgical retractor. In one embodiment, the configuration of the channel of the retaining member is adapted to provide maximum stability for the retaining member during slidable engagement with the shaft of the surgical retractor.

The channel portion (16C) of the retaining member as well as the cross section (12C) of the shaft portion of the surgical retractor may utilize any number of configurations including, but not limited to, octagonal, hexagonal, rectangular, cylindrical, triangular, and/or hourglass configurations. In one embodiment, the interior channel of the retaining member and the cross section (12C) of the shaft portion utilize a “flat” or horizontally elongated configuration wherein the width of the cross section of the shaft portion is greater than its height. This feature of the present invention prevents rotational motion between the retaining member and the shaft, allows linear motion there between, and provides torsional rigidity to the shaft. Thus providing exceptional stability during use of the surgical retractor. This feature also makes it less likely that the vertical profile of the shaft portion of the surgical retractor will act as an obstruction to the surgical personnel during a procedure.

The retaining member (16) of the present invention is extremely versatile and may be utilized with any number of surgical devices. For example, the lower surface of the retaining member may be equipped with an adhesive portion adapted to releasably attach to a surgical drape. The retaining member may be equipped with one or more clips for releasably attaching to a surgical drape or retractor plate. The retaining member may also be equipped with one or more interlocking members (16I), fin members (16F), and/or protrusions (16P) for releasably attaching to a retractor plate (18), as described further below.

In one embodiment, the present invention provides one or more elastic members (20) capable of working in combination with the retaining member (16) and a surgical retractor. In one embodiment, the elastic member is adapted to releasably attach to the retaining member at one end (20B) and releasably attach to the blade or shaft portion of the surgical retractor at the other end (20A). The retractor of the present invention may also provide one or more guides (24) for positioning at least a portion of the elastic member adjacent to the retractor.

In one embodiment, the retaining member may be releasably attached to a surgical drape, a retractor plate, or other surgical apparatus. The shaft portion of the surgical retractor may be inserted into one or more channels provided by the retaining member. The blade portion may be inserted into a body cavity of the patient. A first end (20A) of one or more elastic members may be releasably attached to the blade or shaft portion of the surgical retractor. Tissue may be retracted by the blade portion of the surgical retractor. A second end (20B) of one or more elastic members may then be releasably attached to the retaining member. The force of retracting the tissue places the elastic member in a state of tension such that the blade portion is held in a retracted position by the elastic member as dictated by surgical personnel.

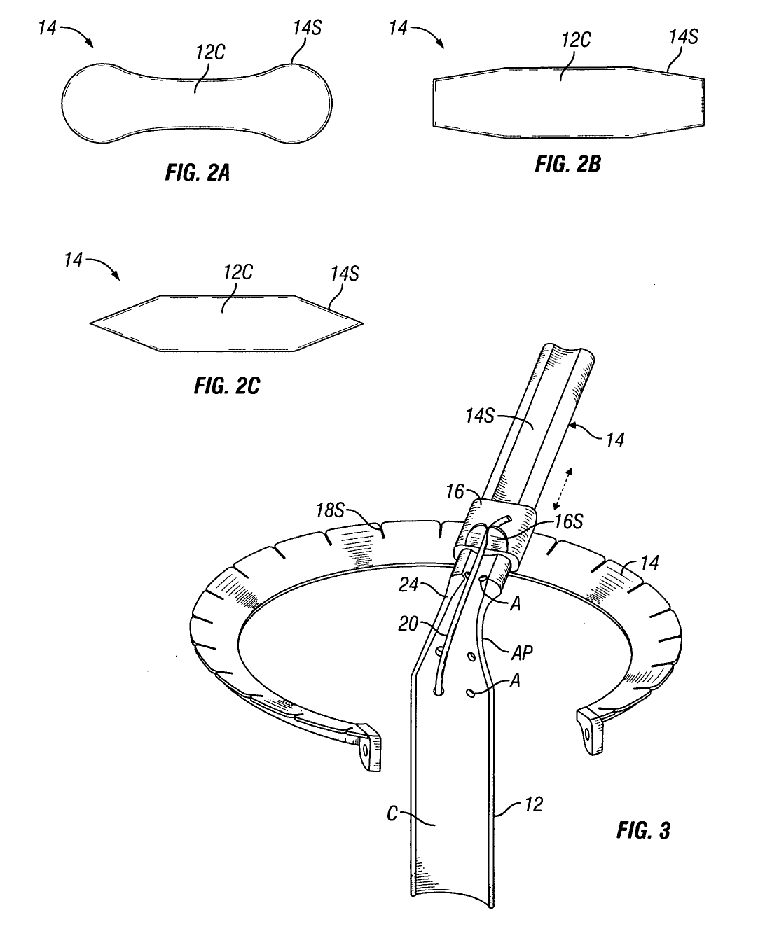

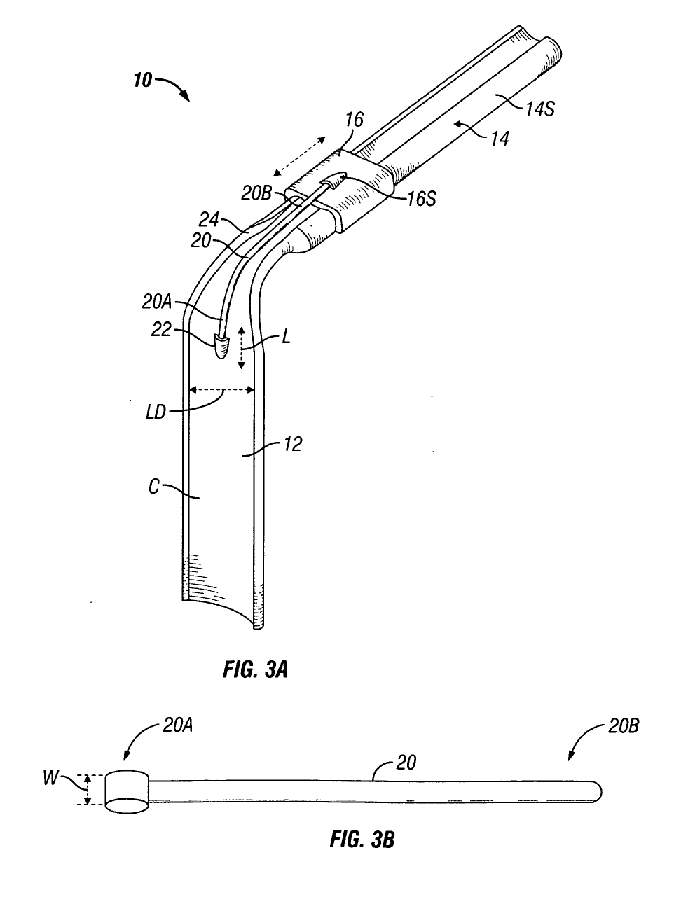

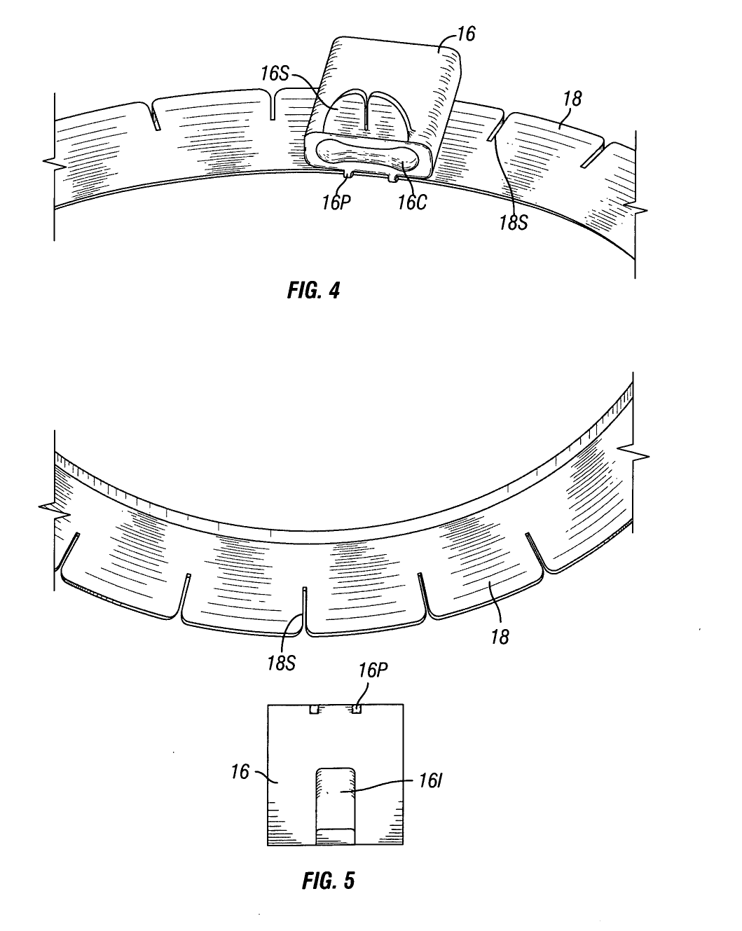

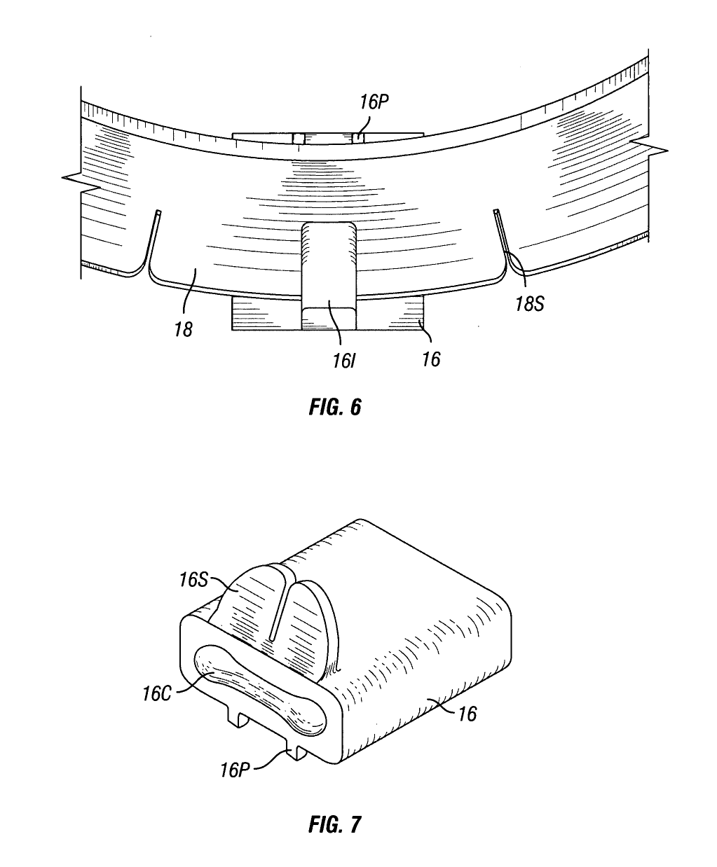

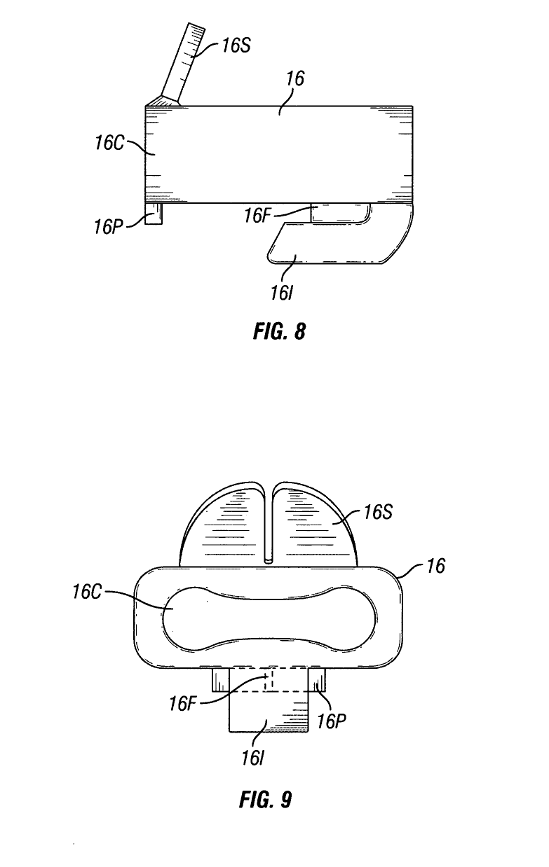

Referring to FIGS. 2, 3, and 3A, in one embodiment, a retractor securing member (22) is utilized to retain a first end of the elastic member (20A) and a securing member (16S) provided upon the retaining member (16) is utilized to retain a second end of the elastic member (20B). FIGS. 2 and 3 illustrate the use of aperture (A) as the securing member, as discussed further below.

The retaining member (16) may provide any number of features capable of receiving and securing one end of the elastic member. The retaining member (16) may provide one or more securing members (16S) for receiving and securing one end of the elastic member (20B) to the retaining member. For example, the securing member may provide a hook, protrusion, clasp, slot, clip, aperture, adhesive or any member capable of receiving and retaining at least one end of the elastic member. The securing member (16S) provided by the retaining member (16) may provide for releasable or permanent attachment of one end of the elastic member. For example, the securing member (16S) may provide an over-molded plastic member or other suitable apparatus such that one end of the elastic member is pre-attached to the retaining member without any action by the surgeon or medical personnel during a procedure.

FIGS. 2 and 3 provide example embodiments wherein the invention utilizes a securing member (16S) to allow for the releasable attachment of one end of an elastic member to the retaining member. FIG. 3A provides an example embodiment wherein the invention utilizes a securing member (16S) to allow for the permanent or pre-attachment of one end of the elastic member to the retaining member.

The surgical retractor (10) may also provide one or more retractor securing members (22) for receiving and securing one end of the elastic member (20A) to the retractor. For example, the retractor securing member (22) may comprise a hook, protrusion, clasp, slot, clip, aperture, adhesive or any member capable of receiving and retaining one end of the elastic member. The retractor securing member (22) may provide for releasable or permanent attachment of one end of the elastic member. For example, the retractor securing member may provide an over-molded plastic member or other suitable apparatus such that one end of the elastic member is pre-attached to the retractor without any action by the surgeon or medical personnel during a procedure.

FIGS. 2 and 3 provide example embodiments wherein the invention utilizes aperture (A) as the securing member to allow for the releasable attachment of one end (20A) of the elastic member to the retaining member. FIG. 3A provides an example embodiment wherein the invention utilizes a securing member (22) to allow for the permanent or pre-attachment of one end of the elastic member (20A) to the blade or shaft portions of the retractor. The retractor securing member (22) may also be adjustable so as to allow the surgeon or medical personnel to adjust the relative position (and correspondingly the attachment point of the elastic member to the retractor) of the retractor securing member upon the retractor. In one embodiment, the retractor securing member (22) may be adjusted in the longitudinal direction (as indicated by arrow (L)) or in the lateral direction (as indicated by arrow LD) via any suitable adjustment mechanism.

Although FIG. 3A illustrates an example embodiment where both ends of the elastic member are pre-attached to their respective securing members, it should be understood that this example in no way limits the scope of the present invention. Specifically, the invention provides embodiments where: (1) one end (either the first end or the second end) of the elastic member is pre-attached, (2) both ends of the elastic member are pre-attached, and (3) neither end of the elastic member is pre-attached, i.e., each end is releasably attachable as described herein.

As discussed above, the blade portion of the surgical retractor provides one or more apertures (A) for receiving at least one end of the elastic member. The shaft portion may also be equipped with one or more apertures for this purpose. In one embodiment, the width (W) of at least a portion of the elastic member is equal to or less than that of at least one of the apertures to allow insertion there through. In this embodiment, a second portion of the elastic member has a width (W) greater than that of one or more of the aperture(s). This feature of the present invention allows a first portion of the elastic member to be inserted into the aperture while the second portion (having the greater width) prevents the elastic member from being removed from the aperture.

In one embodiment, the surgical retractor of the present invention may be utilized in conjunction with a retractor plate. In one embodiment, the invention provides a retractor plate having at least one adhesive portion on its lower surface. The retractor plate of the present invention may be releasably attached to the upper surface of a surgical drape or otherwise secured to any suitable surface.

In one embodiment, the retractor plate of the present invention may utilize one or more grasping members capable of releasably attaching the plate to a surgical drape. In one embodiment, this may involve a generally sliding motion to interlock the plate into one or more grasping members upon or within the drape. In one embodiment, a snap fit arrangement may be provided such that the plate is allowed to snap into one or more receiving members upon or within the drape. The grasping member(s) upon or within the drape may be composed of any suitable rigid, semi-rigid, or soft material capable of successfully interacting with the plate. Further, such members may be constructed of material(s) that are heterogeneous or homogeneous with the drape material. This connection may also utilize one or more adhesive(s). As with the grasping members described above, snap fit arrangement may be releasably or permanently attached to the upper surface of the drape.

It being understood that the retractor plate of the present invention may comprise any number of shapes including, but not limited to, circular, triangular, or non-symmetrical configurations. The shape of the retractor plate may also be altered to correspond to the contour of the bodily region of the patient to be operated on. Further, the retractor plate of the present invention may be composed of any suitable material including semi-pliable or malleable materials. Suitable semi-pliable materials include materials that are capable of being molded into a particular shape while at the same time possessing enough resistance to shape changes to allow proper tension to be exerted on the retractor plate by an elastic stay or blade. Such materials include, but are not limited to, certain plastics and/or malleable metals such as nitinol.

In one embodiment, the retractor plate provides one or more slots (18S) for receiving at least a portion of one or more retaining members. In one embodiment, a lower surface of the retaining member has at least one fin member (16F) for engaging one or more slots (18S) positioned upon the periphery of the retractor plate (18). In this manner, the retaining member may be removably attached to the retractor plate so as to firmly secure the retaining member prior to attachment of the elastic member. In one embodiment, the retaining member has one or more interlocking members (16I) designed to engage the retractor plate. In one embodiment, the interlocking members (16I) and the fin member (16F) may work in concert to securely attach the retaining member (16) to the retractor plate (18). In one embodiment, the retaining member also provides one or more protrusions (16P) for engaging at least a portion of the retractor plate. This feature of the present invention further adds to the stable attachment of the retaining member to the retractor plate.

In one embodiment, the surgical retractor of the present invention may utilize an angular portion (AP) providing for an angle between the shaft portion and the blade portion of between about 45 degrees and about 150 degrees. The angular portion of the present invention may be applied to either the blade or shaft portion, depending on design requirements and the type of surgical procedure. In one embodiment, the angular portion is provided at a first end of the shaft portion and is adapted to provide an angle of between about 45 degrees and about 150 degrees. In one embodiment, the angle formed by the shaft and blade portions of the surgical retractor is about 120 degrees when used in conjunction with a retractor plate and about 100 degrees when used without a retractor plate.

The present invention may be provided to the consumer in any number of ways including, but not limited to, a surgical kit providing two or more retractor blades (12) wherein each blade has at least one corresponding elastic member (20) having a first end attached thereto. A retractor shaft (14) may also be provided and may be attachable/detachable to each provided blade, thus providing any number of possible shaft/blade combinations for use during surgical procedures.

Although the invention has been described with reference to specific embodiments, this description is not meant to be construed in a limited sense. Various modifications of the disclosed embodiments, as well as alternative embodiments of the invention, will become apparent to persons skilled in the art upon reference to the description of the invention. It is, therefore, contemplated that the appended claims will cover such modifications that fall within the scope of the invention.

What is claimed is:

1. A surgical system comprising:
a surgical retractor having a blade portion for retracting tissue and a shaft portion shaped to slideably engage an inner surface of a retaining member; and
an elastic member having a first end for interfacing with said blade portion of said retractor and a second end for interfacing with said retaining member.

2. The surgical system of claim 1, wherein said inner surface of said retaining member forms an interior channel having a configuration selected from the group consisting of: generally hourglass, generally rectangular, generally hexagonal, and generally octagonal.

3. The surgical system of claim 1, wherein said retractor further comprises an aperture for releasably engaging said first end of said elastic member.

4. The surgical system of claim 3, wherein said elastic member has a first portion having a width substantially equal to a width of said aperture and a second portion having a width greater than said width of said aperture.

5. The surgical system of claim 1, wherein said retractor further comprises a retractor securing member for receiving said first end of said elastic member.

6. The surgical system of claim 5, wherein said retractor securing member is selected from the group consisting of a hook, clasp, clip, slot, aperture, protrusion, and adhesive.

7. The surgical system of claim 5, wherein the position of said retractor securing member is adjustable.

8. The surgical system of claim 1, wherein said first end of said elastic member is pre-attached to said retractor or to said retractor securing member.

9. The surgical system of claim 1, wherein said retaining member further comprises a securing member for receiving said second end of said elastic member.

10. The surgical system of claim 9, wherein said securing member is selected from the group consisting of a hook, clasp, clip, slot, aperture, protrusion, and adhesive.

11. The surgical system of claim 1, wherein said second end of said elastic member is pre-attached to said securing member or to said retractor.

12. The surgical system of claim 1, further comprising a retractor plate adapted to overlay the body of the patient, wherein said retaining member is adapted for use with said retractor plate.

13. The surgical system of claim 1, wherein a cross section of at least a portion of said retractor has a horizontally elongated configuration and said inner surface of said retaining member forms an interior channel having a horizontally elongated configuration.

14. The surgical system of claim 1, wherein said retractor further comprises a guide for positioning at least a portion of said elastic member adjacent to said retractor.

15. A surgical system comprising:
a surgical retractor having a blade portion for retracting tissue and a shaft portion shaped to slideably engage an inner surface of a retaining member;
an elastic member having a first end for interfacing with said blade portion of said retractor and a second end for interfacing with said retaining member, said retractor further comprising a retractor securing member for receiving said first end of said elastic member, said retaining member further comprising a securing member for receiving said second end of said elastic member; and
wherein a cross section of at least a portion of said retractor has a horizontally elongated configuration and said inner surface of said retaining member forms an interior channel having a horizontally elongated configuration.

16. The surgical system of claim 15, wherein said retractor securing member is selected from the group consisting of a hook, clasp, clip, slot, aperture, protrusion, and adhesive.

17. The surgical system of claim 15, wherein said retractor securing member is adjustable.

18. The surgical system of claim 15, wherein said retractor further comprises a guide for positioning at least a portion of said elastic member adjacent to said retractor.

19. The surgical system of claim 15, wherein said first end of said elastic member is pre-attached to said retractor or to said securing member.

20. The surgical system of claim 15, wherein said second end of said elastic member is pre-attached to said securing member or to said retractor.
